(19) United States
(12) Patent Application Publication
(10) Pub. No.: US 2014/0004573 A1
(43) Pub. Date: Jan. 2, 2014

(54) PROCESSING BIOMASS

(71) Applicant: Xyleco, Inc., Woburn, MA (US)

(72) Inventors: Marshall Medoff, Brookline, MA (US); Thomas Craig Masterman, Brookline, MA (US); Randy Lavigne, Seabrook, NH (US); Jamie K Huang, Chesterfield, MO (US); Kaitlyn Creasey, Somerville, MA (US)

(21) Appl. No.: 13/932,814

(22) Filed: Jul. 1, 2013

Related U.S. Application Data
(60) Provisional application No. 61/667,156, filed on Jul. 2, 2012.

(51) Int. Cl. C12P 19/14; C12P 19/02

(52) U.S. Cl. CPC C12P 19/14; C12P 19/02
USPC 435/99

(57) ABSTRACT

Biomass (e.g., plant biomass, animal biomass, and municipal waste biomass) is processed to produce useful intermediates and products, such as energy, fuels, foods or materials. For example, systems are described that can use feedstock materials, such as cellulosic and/or lignocellulosic materials, to produce an intermediate or product, e.g., by enzymatic saccharification in a continuous, semi-continuous or non-continuous fashion.

### RELATED APPLICATIONS

This application claims priority to U.S. Provisional Application Ser. No. 61/667,156 filed Jul. 2, 2012, the entire disclosure of which is hereby incorporated by reference herein.

### BACKGROUND OF THE INVENTION

Cellulosic and lignocellulosic materials are produced, processed, and used in large quantities in a number of applications. Often such materials are used once, and then discarded as waste, or are simply considered to be waste materials, e.g., sewage, bagasse, sawdust, and stover.

### SUMMARY

This invention relates to carbohydrate-containing materials (e.g., biomass materials or biomass-derived materials), methods of processing such materials, and intermediates and products resulting from such processing, such as fuels and/or other products. Generally, biomass includes cellulose, hemicellulose, and lignin along with lesser amounts of proteins, extractables and minerals. The complex carbohydrates contained in the cellulose and hemicellulose fractions can be processed into sugars by saccharification, e.g., using cellulolytic enzymes, acid (such as a weak or dilute mineral acid) or acid treatment followed by cellulolytic enzymes, and the sugars can then be used as an end product or intermediate, or converted by further bioprocessing or chemical means e.g., fermentation or hydrogenation, into a variety of products, such as alcohols, sugar alcohols, organic acids and hydrocarbons. The product produced often depends upon the microorganism or chemicals utilized and the conditions under which the processing occurs.

Generally, the invention relates to processes and systems of enhancing saccharification e.g., of biomass material for saccharifying biomass, e.g., cellulosic or lignocellulosic feedstock, in a continuous, semi-continuous or non-continuous manner. Saccharification can be enhanced, e.g., by increasing the overall sugar yield. Without being bound to any particular theory, it is believed that the methods disclosed herein increase saccharification effectiveness by being more cost effective and having less process variability (e.g., less viscosity variability, temperature variability and/or pH variability during the process) while being flexible and allowing high throughput.

In one aspect, the invention features methods of processing biomass materials that include saccharifying a saccharified material. The saccharified material prior to saccharification can be treated by any method described herein, e.g., treated with electron beam radiation.

In another aspect, the invention features a method of processing a cellulosic material, that includes saccharifying a biomass material in a first saccharification tank and a second saccharification tank. In some instances, the first saccharification tank is in fluid communication with the second saccharification tank. The contents of the second saccharification tank have a higher sugar concentration than the contents of the first saccharification tank, for example, the concentration of sugars in the first saccharification tank can be less than about 1 g/L (e.g., less than 5 g/L, less than about 10 g/L, less than about 50 g/L, less than about 100 g/L, less than about 200 g/L, less than about 300 g/L, less than about 500 g/L) and the concentration of sugars in the second saccharfication tank can be at least 1 g/L (e.g., at least 5 g/L, at least 10 g/L, at least 50 g/L, at least 100 g/L, at least 200 g/L, at least 300 g/L, at least 500 g/L). Optionally, the first saccharification tank is in continuous fluid communication with the second saccharification tank. An enzyme, such as one that digests biomass into sugars, can be added to the first saccharification tank during saccharification, and a biomass can be added to the second tank during saccharification.

In another aspect of the invention, the fluid communication between the two tanks can be provided by a fluid flow path between the first saccharification tank and the second saccharification tank. A first separator, can be positioned along the fluid flow path and spent biomass having a carbohydrate level lower than the feedstock biomass material is collected, e.g., for energy generation, on the first separator, while a remaining first supernatant sugar solution flows through the separator into the second tank. A second separator can be positioned along the fluid flow path and a second supernatant sugar solution is collected after passing through the second separator, and biomass filtered out by the second separator is added to the first saccharification tank. The separators can be a mesh, a screen, vibratory screener, a strainer, a centrifuge, a filter, a settling tank or combinations thereof.

Optionally, the temperature in the first and second saccharification tanks is more than about 45° C. (e.g., more than about 55° C., between 45 and 65° C., between 50 and 60° C.).

The biomass can include cellulosic or lignocellulosic material, for example, paper, paper products, paper waste, wood, particle board, sawdust, agricultural waste, sewage, silage, grasses, straw, wheat straw, rice hulls, bagasse, cotton, jute, hemp, flax, bamboo, sisal, abaca, straw, corn cobs, corn stover, alfalfa, hay, coconut hair, seaweed, algae, or mixtures thereof.

In some implementation of the method, the biomass material is mechanically treated, for example, by comminuting, (e.g., cutting, milling, wet milling, freezer milling, hammermilling, pressing, grinding, shearing and chopping). Mechanical treatment can reduce the bulk density of the feedstock and/or increase the surface area of the feedstock. In some embodiments, after mechanical treatment the material has a bulk density of less than 0.75 g/cm³ (less than 0.70 g/cm³, less than 0.65 g/cm³, less than 0.60 g/cm³, less than 0.55 g/cm³, less than 0.50 g/cm³, less than 0.45 g/cm³, less than 0.40 g/cm³, less than 0.45 g/cm³, less than 0.40 g/cm³, less than 0.35 g/cm³, less than 0.30 g/cm³, less than 0.25 g/cm³, less than 0.20 g/cm³, less than 0.15 g/cm³, less than 0.10 g/cm³, less than 0.05 g/cm³). Bulk density is determined using ASTM D1895B.

The biomass, comprising cellulosic or lignocellulosic material can also be treated by radiation, sonication, pyrolysis, oxidation, steam explosion, and combinations of these. These treatment methods can reduce the recalcitrance of the material relative to the recalcitrance of the native material, making the biomass easier to subsequently saccharify. The radiation treatment can be by one or more electron beams. The total dosage of irradiation can be between about 10 Mrad and 200 Mrad. The treatment can include any one or more of the treatments disclosed herein, applied alone or in any desired combination, and applied once or multiple times.

The sugars produced by the saccharification of the disclosed methods can include glucose, xylose, fructose, arabinose, mannose as well as di, tri and poly saccharides. The sugars can be converted to products using an organism, an enzyme or a catalyst.

In one implementation, the methods include processing a cellulosic or lignocellulosic material, by adding an enzyme and a liquid, such as water, to a first saccharification tank, and adding a biomass material to a second saccharification tank. The first saccharification tank is in fluid communication with the second saccharification tank the contents of the second saccharification tank have a higher sugar concentration than the contents of the first saccharification tank.

In yet another aspect, the invention is a system for saccharifying biomass using a first saccharification tank containing a first saccharified material and a second saccharification tank containing a second saccharified material. The first and second saccharified materials are in fluid communication. The first saccharified biomass has a lower concentration of sugar than the second saccharified material. Optionally, the fluid communication is continuous. The system can also include a first separator positioned between the first and second saccharification tanks along a fluid flow path, this fluid flow path providing fluid communication between the first and the second tank. The system can further include a second separator positioned between the first and second saccharification tanks along the fluid flow path. The separators can be any one or more of a mesh, a screen, a vibratory screener, a strainer, a centrifuge, a filter or a settling tank.

The systems for sacchrifying biomass can include a first and a second saccharification tank. A first fluid flow path provides a first fluid communication from the first tank to the second tank. A first separator is disposed in the first fluid flow path for removing processed biomass from fluid communication between the first and second tanks. A second fluid flow path provides a second fluid communication from the second tank to the first tank. A second separator is disposed in the second fluid flow path for removing a saccharified supernatant from fluid communication between the first and second tanks. The system includes a first delivery device configured to add a liquid feedstock to the first tank at about the same rate as the second separator removes saccharified supernatant. The system also includes a second delivery device configured to add a biomass feedstock to a second tank at about the same rate as the second separator removes the processed biomass. Optionally the first fluid flow path and the second fluid flow path provide a constant flow of fluid between the first saccharification tank and second saccharification tank.

Other features and advantages will be apparent from the following detailed description, and from the claims.

### DESCRIPTION OF THE DRAWING

FIG. 1 is a diagram illustrating the enzymatic hydrolysis of cellulose to glucose.

FIG. 2 is a diagram illustrating the action of cellulase on cellulose and cellulose derivatives.

FIG. 3 is a flow diagram illustrating conversion of biomass containing cellulosic or lignocellulosic material to one or more products.

FIG. 4 is a diagram illustrating a method for the saccharification of biomass using two tanks and two separators.

FIG. 5 is a diagram illustrating a method for the saccharification of biomass using four or more tanks and separators.

FIG. 6 shows a particular embodiment of the invention using two tanks and two separators. FIG. 6A is an expanded cutout view of a baghouse. FIG. 6B shows an expanded partial cut out view of a vibratory screener with two screens. FIG. 6C shows an expended cut out view of a vibratory screener with one screen.

FIG. 7 is a flow diagram illustrating a method for saccharification of biomass in a non-continuous fashion.

### DETAILED DESCRIPTION

Using the methods described herein, biomass (e.g., plant biomass, animal biomass, paper, and municipal waste biomass) can be processed to produce sugars and other useful intermediates and products such as organic acids, salts of organic acids, anhydrides, esters of organic acids and fuels, e.g., fuels for internal combustion engines or feedstocks for fuel cells. Systems and processes are described herein that include continuous, semi-continuous or batch processing of biomass, for example the continuous saccharification of cellulosic or lignocellulosic material using two or more tanks and separators.

In order to convert the feedstock to a form that can be readily processed, the glucan-or xylan-containing cellulose in the feedstock is hydrolyzed to low molecular weight carbohydrates, such as sugars, by a saccharifying agent, e.g., an enzyme or acid, a process referred to as saccharification. The low molecular weight carbohydrates can then be used, for example, in an existing manufacturing plant, such as a single cell protein plant, an enzyme manufacturing plant, or a fuel plant, e.g., an ethanol manufacturing facility.

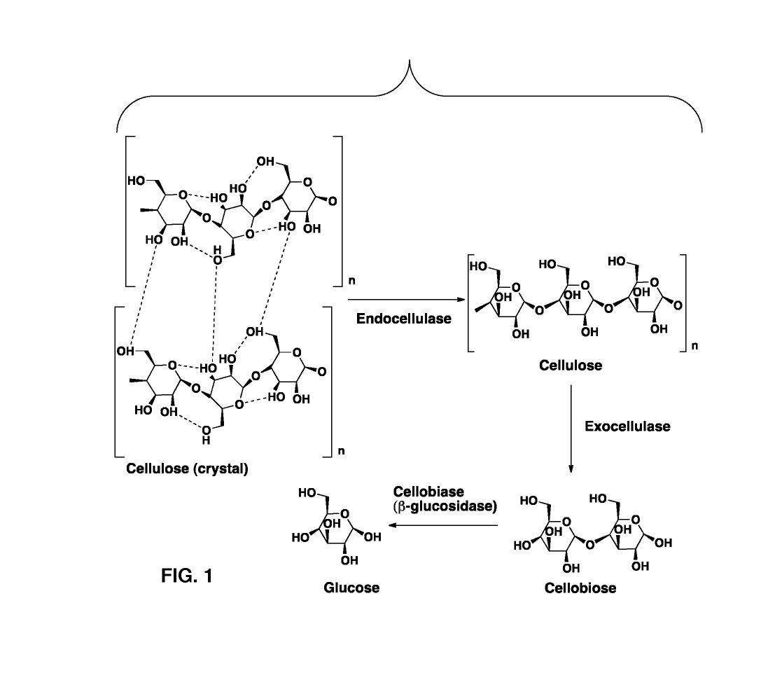

Enzymes and biomass-destroying organisms that break down biomass, such as the cellulose and/or the lignin portions of the biomass, contain or manufacture various cellulolytic enzymes (cellulases), ligninases or various small molecule biomass-destroying metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (β-glucosidases). Referring to FIG. 1, during saccharification a cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose.

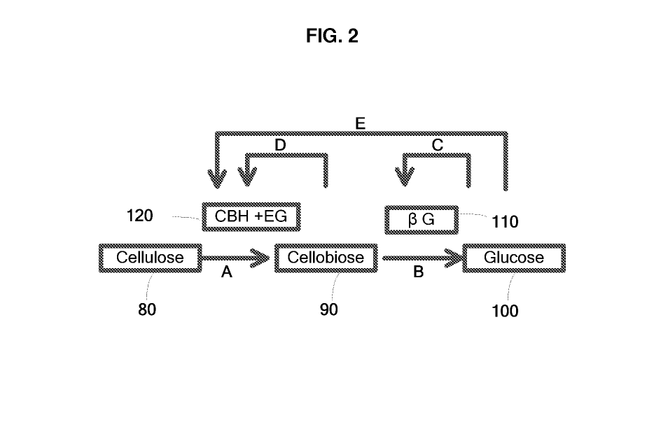

Referring now to FIG. 2, hydrolysis of cellulose (80) is a multi-step process which includes initial breakdown at the solid-liquid interface via the synergistic action of endoglucanaes (EG) and exo-glucanaes/cellobihydolases (CHB) (step A) (120). This initial degradation is accompanied by further liquid phase degradation by hydrolysis of soluble intermediate products such as oligosaccharides and cellobiose (90) that are catalytically cleaved by β-glucosidase (βG, 110) in (step B). Cellobiose directly inhibits both CBH and EG (120) as indicated in (step D). Glucose (100) directly inhibits βG (110) (step C), CBH and EG (120) (step E). The methods described herein can eliminate or reduce this inhibition, providing much higher yields of sugar. In addition or in combination, with the methods described herein, contacting the feedstock with the additives, for example glucose isomerase, can also reduce or eliminate this inhibition (steps C and E) as described in PCT/US12/71093 and PCT/US 12/71097 both written in English and filed on Dec. 20, 2012 the entire disclosures of which are incorporated herein by reference.

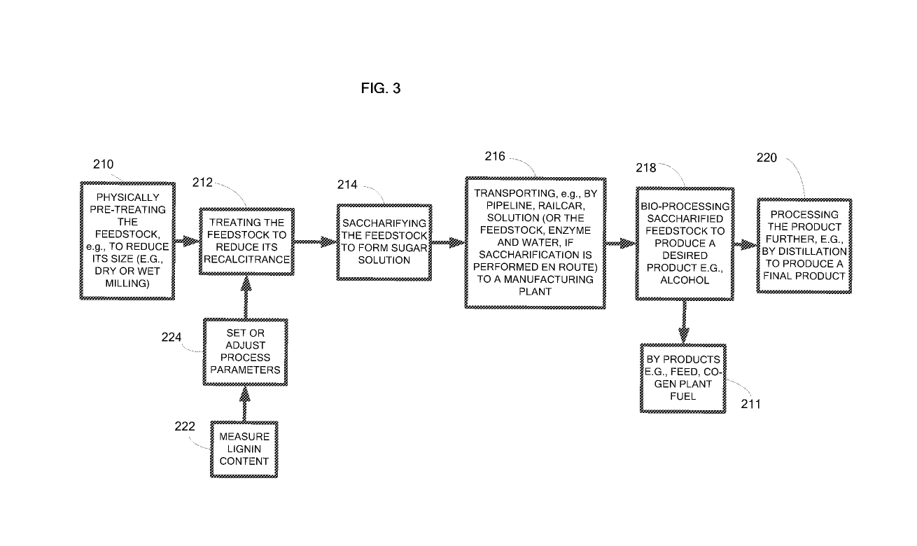

Biomass that has been saccharified by the methods described herein can be manufactured into various products, for example, by reference to FIG. 3, showing a process for manufacturing an alcohol. The method can include, for example, optionally mechanically treating a feedstock (step 210), before and/or after this treatment, optionally treating the feedstock with another physical treatment, for example irradiation, to further reduce its recalcitrance (step 212), and saccharifying the feedstock, using the methods described herein, to form a sugar solution (step 214). Optionally, the method may also include transporting, e.g., by pipeline, railcar, truck or barge, the solution (or the feedstock, enzyme and water, if saccharification is performed en route) to a manufacturing plant (step 216). In some instances the saccharified feedstock is further bioprocessed (e.g., fermented) to produce a desired product (step 218) and byproduct (211). The resulting product may in some implementations be processed further, e.g., by distillation (step 220). If desired, the steps of measuring lignin content (step 222) and setting or adjusting process parameters based on this measurement (step 224) can be performed at various stages of the process, as described in U.S. Pat. No. 8,415,122 filed Feb. 11, 2010 the entire disclosure of which is incorporated herein by reference.

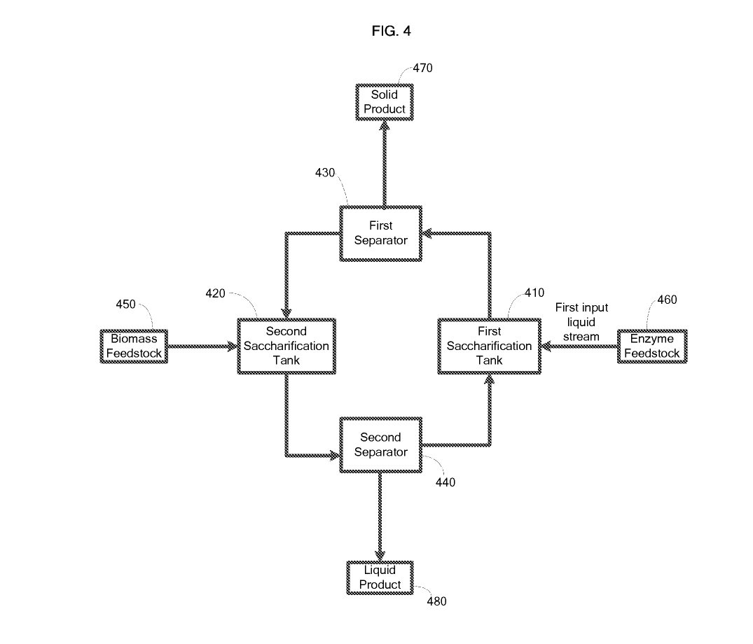

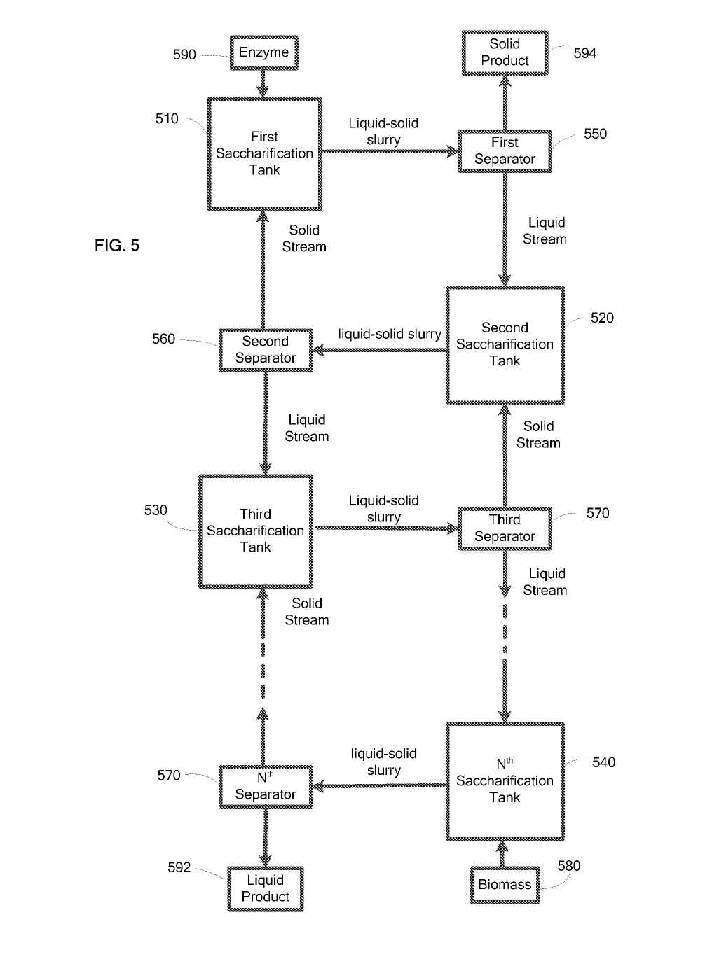

Referring to FIG. 4, a method for saccharifying a feedstock biomass material (e.g., cellulosic or lignocellulosic material) is shown. A first saccharification tank (410) and a second saccharification tank (420) are in fluid communication through a first separator (430) and a second separator (440). Biomass feedstock (450) can be added to the second tank (420) and an enzyme feedstock (460) can be added to the first tank (410). The contents of the first tank (410) are made to flow through the first separator (430). The first separator partitions the saccharifying mixture into a liquid stream, made to flow into the second tank (420), and a solid stream (470), e.g., solid product, spent biomass or processed biomass) that can be collected for further processing. The contents of the second tank (420) are made to flow through the second separator (440). The second separator partitions the saccarifying material from the second tank (420) into a solid stream, that is made to flow into the first tank (410), and a liquid stream (480) (e.g., liquid product, saccharified sugar solution, sugar solution, saccarified supernatant). The concentration of sugars in the saccharifying material in the first tank (410) is less than the concentration of sugars in the saccharifying material in the second tank (420). The amount of extractable sugars in the spent biomass is less than the amount of extractable sugars in the biomass feedstock. Extractable sugars are sugars that may be in a bound, trapped and/or insoluble form. For example, in the form of carbohydrates (e.g., monosaccharides, disaccharides, trisaccharides and/or polysaccharides) in the biomass, adsorbed to surfaces and/or trapped in the biomass.

All or just a portion of the liquids from the first separator can be sent to the second saccharification tank. All or just a portion of the solids from the first separator can be partitioned as solid product, for example a portion of the solids can be sent back to the first saccarification tank. In some cases the portion that is sent back to the first saccharification tank has a larger average particle size than the potion sent as solid product. All or just a portion of the solids from the second separator can be sent to the second saccharification tank. In some cases the portion that is sent back to the second saccharification tank has larger average particle size than the portion that is sent to the first saccharification tank. All or just a portion of the liquids from the second separator can be collected as liquid product.

As further shown by FIG. 4, each tank is in fluid communication with two separators. In other optional configurations, each tank may be in fluid communication with three or more separators (eg. at least four, at least five, at least 6) with inward or outward fluid flows.

The biomass in the second tank is typically combined with a liquid (e.g., water) prior to and/or after addition to the second tank. For example the biomass may be a substantially dry biomass (e.g., containing less than 25 wt. % water, less than 10 wt. % water or less than 5 wt. % water) that is added to the tank using a conveyor (e.g., belt or vibratory), extruder, air blower, a hopper and/or manually. In options wherein the biomass is combined or already contains water prior to addition to the second tank, the biomass can be sent to the second tank using, for example a tube in combination with a pump or using gravity, a liquid screw extruder or any other useful means. Additional water can be added to the second or first tank as needed from a water source such as a water tank connected to a tube and fed to the first tank, second tank, or any other equipment in fluid communication with the tanks, by, for example a pump or gravity, under control of valves that are, for example, either remotely or manually controlled. The solid biomass is typically added to have at least 5 wt. % biomass in the tank (e.g., at least 10 wt. %, at least 20 wt. %, at least 30 wt. %, at least 40 wt. %, at least 50 wt. %, at least 60 wt. %, at least 70 wt. %).

The enzyme feedstock (e.g., cellulase) is added to the first tank, for example, in a liquid form (e.g., dissolved and/or suspended in an aqueous solution). The enzymes can be added to provide a concentration in the first tank, for example, of at least 1 mg enzyme per gram of feedstock (e.g., at least 5 mg/g, at least 10 mg/g, at least 20 mg/g). The enzyme feedstock itself may be in a concentrated form, for example at least 10 mg/mL (e.g., at least 20 mg/ml, at least 40 mg/mL, at least 60 mg/mL, at least 80 mg/L). The enzyme activity in the first and second tank is between about 0.1 and 10 μmol/min/mg (e.g, between about 0.1-1 μmol/min/mg, 0.1-0.8 μmol/min/mg, 0.1-0.6 μmol/min/mg, 0.1-0.4 μmol/min/mg, 0.2-10 μmol/min/mg, 0.2-1 μmol/min/mg, 0.2-0.8 μmol/min/mg, 0.2-0.6 μmol/min/mg, 0.4-1 μmol/min/mg, 0.4-1 μmol/min/mg, 0.6-10 μmol/min/mg, 1 to 10 μmol/min/mg) use a FP assay (Filter paper assay, Ghose, IUPAC, Measurement of Cellulase Activities, T. K. Ghose; Pure & Appl. Chem., Vol. 59, No. 2, pp. 257, 1987). The enzyme activity in the first and second tank can be between about 0.1 and 40 μmol/min/mg (e.g. 0.1-20 μmol/min/mg, 0.1-10 μmol/min/mg, 0.1-5 μmol/min/mg, 1-40 μmol/min/mg, 1-20 μmol/min/mg, 1-10 μmol/min/mg, 1-8 μmol/min/mg, 1-6 μmol/min/mg, 2-40 μmol/min/mg, 2-20 μmol/min/mg, 2-10 μmol/min/mg, 2-8 μmol/min/mg, 2-6 μmol/min/mg , 6-20 μmol/min/mg) using a CB assay (cellobiase activity).

At any point in the process additives may be added, for example, acids, bases and buffers can be added to control the pH. Surfactants can be added to modify the viscosity, mixing and flow properties of the compositions in the various tanks and equipment. Examples of surfactants include non-ionic surfactants, such as a Tween® 20 or Tween® 80 polyethylene glycol surfactants, ionic surfactants, or amphoteric surfactants. Other suitable surfactants include octylphenol ethoxylates such as the TRITON™ X series nonionic surfactants commercially available from Dow Chemical. A surfactant can also be added to keep the sugar that is being produced in solution, particularly in high concentration solutions. Optionally an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during the saccharification or transport and storage, and can be used at appropriate concentrations, e.g., between 15 and 1000 ppm, e.g., between 25 and 500 ppm, or between 50 and 150 ppm. In addition, chemical sterilization agents can be added to control microbial growth during the processes, gases such as air, nitrogen, argon, carbon dioxide, nitrous oxide, chlorine, oxygen, ozone can be added by bubbling through the liquid solutions or blanketing the saccharification tanks, and glucose isomerase can be added to reduce inhibition of cellulase. Optionally, the pH is maintained between pH 2 and pH 8 (e.g., between pH 3 and pH 6, between pH 3.5 and pH 4.5). The temperature of the saccharifying biomass during the process is preferably between about 30° C. and 70° C. (e.g., between 40° C. and 60° C., eg. between about 45° C. and 55° C.). In some embodiments, the temperature of the saccharifying biomass during the process is above about 40° C. (e.g., above about 45° C., above about 50° C., above about 55° C.). The temperature and pH of the saccharifying biomass may be the same or different in different parts of the equipment, for example in the tanks or in the separators.

In some embodiments liquid product is produced at a rate between about 1 and about 20 tank volumes per day per day (e.g., between about 2 and about 16 tanks per day, between about 4 and about 12 tanks per day). A tank volume refers to the total amount of liquid present in all the tanks used during the process.

The process can be operated in a continuous manner, with an about constant flow of material from the first tank through the first separator, to the second tank, through the second separator, to the first tank, and an about constant addition of enzyme feedstock and biomass feedstock. Thus when used in a continuous manner, the volumes of liquid-biomass slurry in the tanks stays about constant. In one embodiment the flow of materials discussed above is maintained so as to extract at least 50% of the available sugars from the biomass (e.g. at least 40 wt. %, at least 50 wt. %, at least 60 wt. %, at least 70 wt. %, at least 80 wt. % or even at least 90 wt. %). Optionally, the flow as described above is maintained so as to produce a liquid product with at least 5 wt. % sugars (e.g., at least 10 wt. %, at least 20 wt. %, at least 30 wt. %, at least 40 wt. %, at least 50 wt. %). In some embodiments the flow of materials is maintained to produce a solid product wherein up to 50% of the extractable sugars (e.g. carbohydrates) have been removed from the biomass (up to 60 wt. %, up to 70 wt. %, up to 80 wt. %, up to 90 wt. % or even up to 100 wt. %). The process can be operated in an at least partially non-continuous manner (e.g., semi-continuous or even in a batch mode). For example, the first tank (410) can be partially or completely emptied to the first separator (430) at any time during the saccharification process and as many times as desired, for example, to optimize the processing.

In an example of non-continuous operations, at least 10 vol. %, e.g., at least 20 vol. %, at least 30 vol. %, at least 40 vol. %, at least 50 vol. %, at least 60 vol. %, at least 70 vol. %, at least 80 vol. %, or at least 90 vol. %, of the contents of the first tank (410) are sent to the first separator (430) when the saccharification is completed (or at least 20% completed, at least 40% completed, at least 60% completed, at least 80% completed). The saccharification is considered completed at a point wherein saccharification for an additional 8 hours or more will not yield more than 10% more sugars. For example, if the saccharification in the first tank yields 10 wt. % sugars (about 100 g/L), it is considered complete if saccharification for 8 or more additional hours (e.g., using the same, equivalent or similar conditions) will not yield more than 1 wt. % (10 g/L) more sugars. Once some of the saccharified material as described above is fed to the first separator (430), solids from the second separator (440), enzyme feedstock (460) and liquids (e.g. water) can be added to the first tank (410) to provide a volume that is about equal to, less than, or more than the original volume in the first tank (410), for example up to 150 vol % of the tank, up to 120 vol. %, up to 100 vol. %, or at least 90 vol % at least 80 vol. %, at least 70 vol. %, at least 60 vol. %, at least 50 vol. %, at least 40 vol. % at least 30 vol. %, at least 20 vol. % or at least 10 vol. %. All or just a portion of the solids from the second separator (440) can be fed to the first tank (420), for example at least 90 vol. %, at least 80 vol. %, at least 70 vol. %, at least 60 vol. %, at least 50 vol. %, at least 40 vol.%, at least 30 vol. %, at least 20 vol. % or at least 10 vol. %.

As another example of non-continuous operation, the second tank (420) can be partially or completely emptied to feed the second separator (440) at any time during the saccharification process and as many times as desired. For example at least 10 vol. %, e.g., at least 20 vol. %, at least 30 vol. %, at least 40 vol. %, at least 50 vol. %, at least 60 vol. %, at least 70 vol. %, at least 80 vol. %, or at least 90 vol. %, of the contents of the second tank can be sent to the second separator (440) when the saccharification is completed (or at least 20% completed, at least 40% completed, at least 60% completed, at least 80% completed). Once some of the saccharified material as described above is fed to the second separator (440), liquids from the first separator (430), biomass (450) and additional liquids (e.g., water) can be added to the second tank (420) to provide a volume that is about equal to, less than, or more than the original volume in the tank, for example up to 150 vol % of the tank, up to 120 vol. %, up to 100 vol. %, or at least 90 vol % , at least 80 vol. %, at least 70 vol. %, at least 60 vol. %, at least 50 vol. %, at least 40 vol. % at least 30 vol. %, at least 20 vol. % or at least 10 vol. %. All or just a portion of the liquids from the first separator (430) can be fed to the second tank (420), for example at least 90 wt. %, e.g., at least 80 wt. %, at least 70 wt. %, at least 60 wt. %, at least 50 wt. %, at least 40 wt. %, at least 30 wt. %, at least 20 wt. % or at least 10 wt. %.

The separators used in the methods and systems described herein can be any useful separator for providing at least two streams from the saccharification tanks. For example the separators can be any one or more of a centrifuge, a filtering device (e.g., gravity, vacuum, filter press, filter bag, porous container) and a settling tank. Additionally, for example, the separators may include a porous material, a mesh, a strainer, a vibratory screener, a perforated plate or cylinder, a strainer, a sieving device, and may have average opening sizes between ½ inch to 1/256 of an inch e.g., between about ¼ inch to 1/64^{th} inch, less than about 0.79 mm ( 1/32 inch, 0.03125 inch), e.g., less than about 0.40 mm (1/64 inch, 0.015625 inch), less than about 0.20 mm ( 1/128 inch, 0.0078125 inch), or even less than about 0.10 mm ( 1/256 inch, 0.00390625 inch). Any combination of separators listed above may be used. In some embodiments the separator is a vibratory screener with one or more screens. The separator produces one or more solid streams, having a higher concentration of solids than the solid concentration of the material in the tank feeding the separator, and one or more a liquid streams, having a lower solid concentration than the concentration of solids in the material in the tank feeding the separator. For example the solid stream would be at least 10 wt. % solids e.g., at least 20 wt. %, at least 30 wt. %, at least 40 wt. %, at least 50 wt. %, at least 60 wt. %, at least 70 wt. %, at least 80 wt. % at least 90 wt. %, or at least 95 wt %. For example the liquids stream would be 1 wt. % or less solids, e.g., 5 wt. % or less, 10 wt. % or less, 20 wt. % or less, 30 wt. % or less, 40 wt. % or less, 70 wt. % or less, 80 wt. % or less, 90 wt. % or less, or 95 wt. % or less.

The tanks used can be in any useful configuration and size. For example the tanks generally would be larger than 100 L (e.g., 400 L, 40,000 L, or 500,000 L). The temperature of the process can be controlled by, for example, temperature controlling jackets and/or insulation on the tanks and tubing.

It is generally preferred that the tank contents be mixed e.g., using jet mixing as described in U.S. Ser. No. 12/782,694 filed on May 18, 2011, Ser. No. 13/293,985 filed on Nov. 10, 2011 and Ser. No. 13/293, 977 filed on Nov. 10, 2011; the full disclosures of which are incorporated herein by reference. For example, in some implementations, one jet mixer is used. In other implementations two or more jet mixers are positioned in the vessel, with one or more being configured to jet fluid upward (“up pump”) and one or more being configured to jet fluid downward (“down pump”). In some cases, an up pumping mixer will be positioned adjacent a down pumping mixer, to enhance the turbulent flow created by the mixers. If desired, one or more mixers may be switched between upward flow and downward flow during processing. It may be advantageous to switch all or most of the mixers to up pumping mode during initial dispersion of the feedstock in the liquid medium, particularly if the feedstock is dumped or blown onto the surface of the liquid, as up pumping creates significant turbulence at the surface.

The solid feedstock (410) can be disposed in one or more porous containers, e.g., a bag or other structure made of mesh or other porous material. For example, a biomass feedstock can be disposed in a carrier as described in PCT/US12/71091filed Dec. 20, 2012. the entire disclosure of which is herein incorporated by reference. Optionally, the container containing biomass can be moved from the first saccharification tank (420), then to second saccharificaton tank (410) and finally removed to provide a spent material in the container during processing. In this case, the container is a separator.

The liquid product (480) and solid product (470) can be further processed for example, to make intermediates and products, as discussed below.

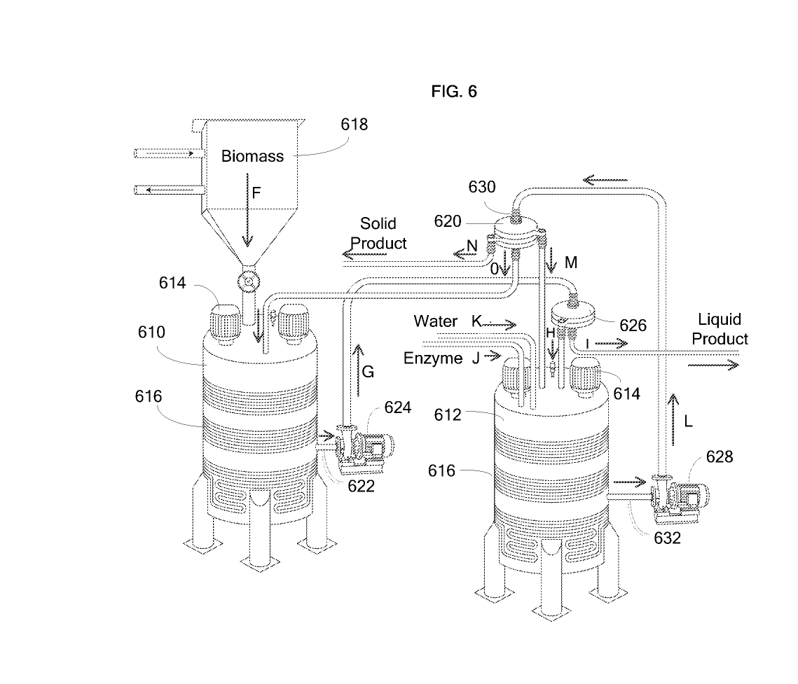

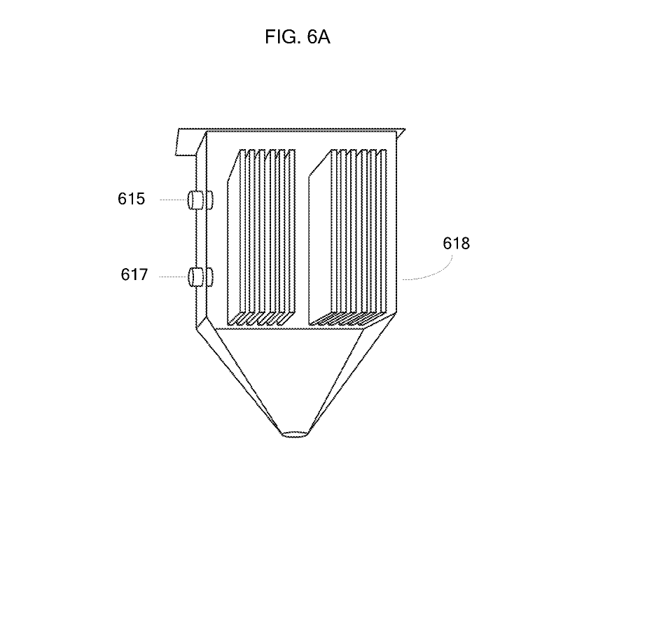

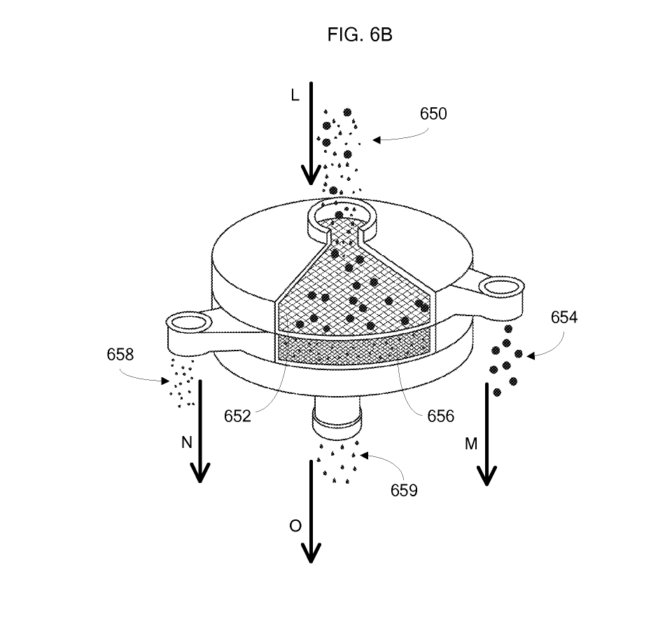

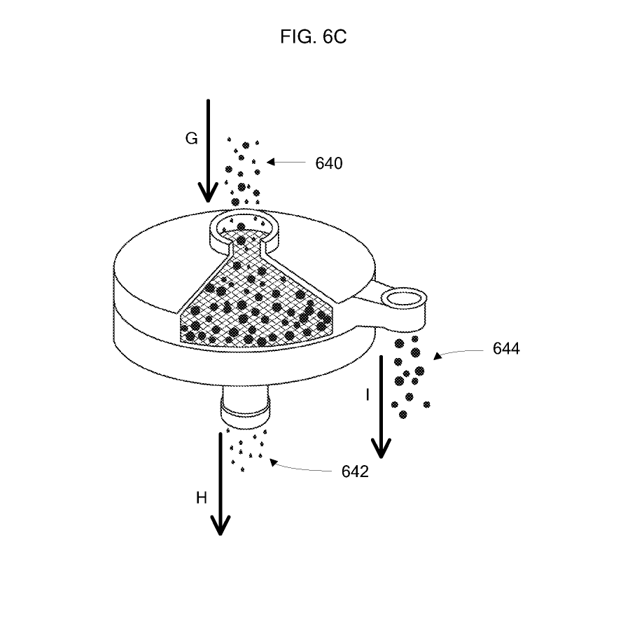

In some embodiments three or more tanks can be used. For example, FIG. 6 is shows an embodiment wherein, a process for saccharifying a biomass (e.g., cellulosic or lignocellulosic material) using four or more saccharification tanks or separators is utilized. The functioning of the tanks and separators are the same as previously described. Therefore, a first saccharification tank (510), a second saccharification tank (520), a third saccharification tank (530) and optionally more saccharification tanks (e.g., up to N tanks (540) where N can be at least 4) are in fluid communication through a first separator (550), a second separator (560), a third separator (570) and optionally more separators (e.g., up to N separators (580) where N can be at least 4). Biomass feedstock (580) can be added to the N^{th} tank (540) and an enzyme feedstock (590) can be added to the first tank (510). Liquid product (592) is provided from the output of the N^{th} separator and solid product (594) is provided from the output of the first separator (550). Using three or more saccharification tanks can provide added advantages over a two tank system with respect to throughput, saccharification efficiency, equipment costs and process stability.

FIG. 6 shows a particular embodiment of the invention utilizing two saccharifiation tanks and two separators. The first tank (610) and the second tank (612) are equipped with two mixing motors (614) that can be removably attached to mixers, e.g. jet mixers, impellors and propellers through a shaft providing mechanical communication from the motor to the mixing head (not shown). The tanks also have a half pipe jacket (616) for temperature control via a flowing fluid such as water. Biomass is conveyed by air using a blower through a bag house (618) to the first tank in the direction shown by arrow F. As depicted in the expanded view of the baghouse, FIG. 6A, the baghouse has an inlet (615) for the biomass and air, and an outlet (617) for the air and some biomass fines. Biomass enters the first tank (610) through a tube connecting the baghouse to the tank port opening. Liquid is supplied from the second tank (612) through a first vibratory screener (620) at a constant rate while liquid-biomass slurry is removed at a comparable rate through an opening connected to a tube (622) on the side of the first tank. The opening for removing of the slurry can be located at different positions on the tank wall and its location can help control the process since the larger, less saccharified, biomass tends to sink lower down into the tank, while more saccharified smaller particles tend to rise up and are more homogeneously dispersed in the tank. The tube for removing the slurry can even extend into the tank, for example from the top so that the opening for removing slurry can be at any position (e.g., vertically and horizontally positioned) in the tank. The slurry is drawn out from the first tank using a pump (624) and sent to a second vibratory screener (626) in the direction shown by arrow G. The second vibratory screener sends solids from the biomass-liquid slurry to a tube that directs the solids to the second tank in the direction of arrow H while the liquid product is passed through second vibratory screener in the direction of arrow I and is collected or sent directly for further processing. Enzyme and water are added to the second tank through two tubes attached to openings at the top of the tank, flowing in the directions of arrows J and K respectively. Liquid-slurry biomass from the second tank (612) is removed at a comparable rate to the addition of fluids (enzyme/water). The liquid-slurry biomass is removed through an opening connected to a tube that is located on the side of the second tank (612). This opening can be located anywhere on the side of the tank and can extend into the tank via a tube, for example, as previously described for the first tank (610). The slurry is drawn out of the second tank through an opening connected to tube (632) using a pump (628) and is conveyed to a first vibratory screener (620) flowing in the direction indicated by arrow L. The first vibratory screener produces three output streams flowing in the direction shown by the arrows M. N and O. The first stream, flowing in the direction indicated by arrow M, is a first solid with a large particle size that is sent back to the second tank for further saccharification. The second stream, flowing in the direction indicated by arrow N, is a second solid with smaller particle size that is collected and/or used for energy production (e.g. co-generation). The third stream, flowing in the direction indicated by arrow O, is a liquid stream that is send to the first tank (610). Connections to the first and second vibratory screeners are made using flexible tubing (630) since the screeners need to oscillate during operation. Supporting structures (not shown) for the vibratory screeners are also flexible. The operation of the screens is now discussed.

FIG. 6B shows a cutout of the first vibratory screener (620). Biomass-liquid slurry (650) flows in the direction indicated by arrow L and enters the screener through an ingress port positioned at the top of the screener. Large particles from the slurry cannot pass through the first screen (652) and move to an egress port on the side of screener (654) and then this stream, the flow direction shown by arrow M, is feed back into the second tank (612). Smaller particles from the slurry pass through the first screen (652) but cannot pass through the second screen (656), which has a smaller mesh size than the first screen. The smaller particles (658) therefore move to an egress port on the side of the screener and are removed as solid product from the system, flowing in the direction indicated by arrow N. The smallest particles (659) and most of the fluid pass through the second screen (656) and are fed to the first tank, flowing in the direction of arrow O. As depicted in FIG. 6C, the second vibratory screener (630) has only one screen and separates the input slurry (640), flowing in the direction of arrow G, into a liquid product (642) flowing in the direction of arrow H and a solid stream (644) flowing in the direction of arrow I.

The saccharification process can be partially or completely performed in) in a manufacturing plant, and/or can be partially or completely performed in transit, e.g., in a rail car, tanker truck, or in a supertanker or the hold of a ship.

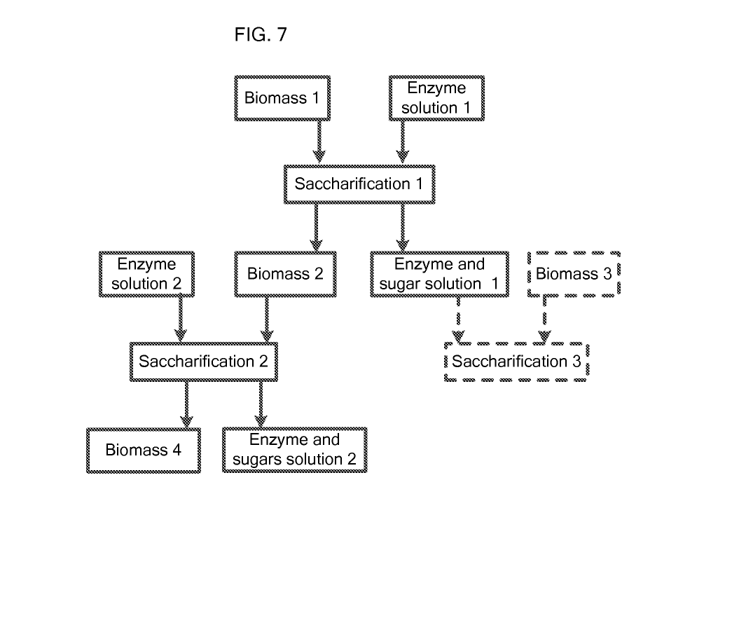

FIG. 7 is a flow diagram illustrating another embodiment of the invention. The embodiment is a method for saccharification of biomass in a non-continuous fashion. A first biomass and first enzyme solution are combined in a tank and a first saccharification occurs. After a desired degree of saccharification has occurred, the biomass (biomass 2) and enzymes and sugars (enzymes and sugars 1) are separated, e.g., with separators such as those discussed herein. The enzyme and sugar solution 1 can then be processed to a product, e.g., a sugar and then optionally other products e.g., alcohols. The enzyme and sugar solution 1 can also be combined with more biomass (e.g., biomass 3) and the biomass saccharified (saccharification 3), optionally wherein more fresh enzyme is added. The biomass 2 can be combined with fresh enzyme solution (enzyme solution 2) and a second saccharification (saccharification 2) can be made to occur. After saccharification 2 is allowed to proceed to the desired degree, the biomass (biomass 4) can separated from the Enzyme and sugar (enzyme and sugars solution 2). The enzyme and sugars solution 2 can then be processed to a product.

### Physical Treatment of Feedstock

### Physical Preparation

In some cases, methods can include a physical preparation, e.g., size reduction of materials, such as by cutting, grinding, shearing, pulverizing or chopping. For example, material can be first pretreated or processed using one or more of the methods described herein, such as radiation, sonication, oxidation, pyrolysis or steam explosion, and then size reduced or further size reduced. In other cases, treating first and then size reducing can be advantageous. Screens and/or magnets can be used to remove oversized or undesirable objects such as, for example, rocks or nails from the feed stream. In some cases no pre-processing is necessary, for example when the initial recalcitrance of the biomass is low, and wet milling is sufficiently effective to reduce the recalcitrance, for example, to prepared the material for further processing, e.g., saccharification.

Feed preparation systems can be configured to produce streams with specific characteristics such as, for example, specific maximum sizes, specific length-to-width, or specific surface areas ratios. Physical preparation can increase the rate of reactions or reduce the processing time required by opening up the materials and making them more accessible to processes and/or reagents, such as reagents in a solution. The bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state. The material can be densified, for example from less than 0.2 g/cc to more than 0.9 g/cc (e.g., less than 0.3 to more than 0.5 g/cc, less than 0.3 to more than 0.9 g/cc, less than 0.5 to more than 0.9 g/cc, less than 0.3 to more than 0.8 g/cc, less than 0.2 to more than 0.5 g/cc). For example, the material can be densified by the methods and equipment disclosed in U.S. Pat. No. 7,932,065 and WO 2008/073186, the entire disclosures of which are herein incorporated by reference. Densified materials can be processed by any of the methods described herein, or any material processed by any of the methods described herein can be subsequently densified.

### Size Reduction

In some embodiments, the material to be processed is in the form of a fibrous material that includes fibers provided by shearing a fiber source. For example, the shearing can be performed with a rotary knife cutter.

For example, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process.

For example, a rotary knife cutter can be used to concurrently shear the fiber source and screen the first fibrous material. A rotary knife cutter includes a hopper that can be loaded with a shredded fiber source prepared by shredding a fiber source.

In some implementations, the feedstock is physically treated prior to saccharification and/or fermentation. Physical treatment processes can include one or more of any of those described herein, such as mechanical treatment, chemical treatment, irradiation, sonication, oxidation, pyrolysis or steam explosion. Treatment methods can be used in combinations of two, three, four, or even all of these technologies (in any order). When more than one treatment method is used, the methods can be applied at the same time or at different times. Other processes that change a molecular structure of a biomass feedstock may also be used, alone or in combination with the processes disclosed herein.

### Mechanical Treatments

In some cases, methods can include mechanically treating the biomass feedstock. Mechanical treatments include, for example, cutting, milling, pressing, grinding, shearing and chopping. Milling may include, for example, ball milling, hammer milling, rotor/stator dry or wet milling, freezer milling, blade milling, knife milling, disk milling, roller milling or other types of milling. Other mechanical treatments include, e.g., stone grinding, cracking, mechanical ripping or tearing, pin grinding or air attrition milling.

Mechanical treatment can be advantageous for “opening up,” “stressing,” breaking and shattering the cellulosic or lignocellulosic materials, making the cellulose of the materials more susceptible to chain scission and/or reduction of crystallinity. The open materials can also be more susceptible to oxidation when irradiated.

In some cases, the mechanical treatment may include an initial preparation of the feedstock as received, e.g., size reduction of materials, such as by cutting, grinding, shearing, pulverizing or chopping. For example, in some cases, loose feedstock (e.g., recycled paper, starchy materials, or switchgrass) is prepared by shearing or shredding.

Alternatively, or in addition, the feedstock material can first be physically treated by one or more of the other physical treatment methods, e.g., chemical treatment, radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the molecular structure of the material by mechanical treatment.

In some embodiments, the feedstock material is in the form of a fibrous material, and mechanical treatment includes shearing to expose fibers of the fibrous material. Shearing can be performed, for example, using a rotary knife cutter. Other methods of mechanically treating the feedstock include, for example, milling or grinding. Milling may be performed using, for example, a hammer mill, ball mill, colloid mill, conical or cone mill, disk mill, edge mill, Wiley mill or grist mill. Grinding may be performed using, for example, a stone grinder, pin grinder, coffee grinder, or burr grinder. Grinding may be provided, for example, by a reciprocating pin or other element, as is the case in a pin mill. Other mechanical treatment methods include mechanical ripping or tearing, other methods that apply pressure to the material, and air attrition milling. Suitable mechanical treatments further include any other technique that changes the molecular structure of the feedstock.

If desired, the mechanically treated material can be passed through a screen, e.g., having an average opening size of 1.59 mm or less ( 1/16 inch, 0.0625 inch). In some embodiments, shearing, or other mechanical treatment, and screening are performed concurrently. For example, a rotary knife cutter can be used to concurrently shear and screen the feedstock. The feedstock is sheared between stationary blades and rotating blades to provide a sheared material that passes through a screen, and is captured in a bin.

The cellulosic or lignocellulosic material can be mechanically treated in a dry state (e.g., having little or no free water on its surface), a hydrated state (e.g., having up to ten percent by weight absorbed water), or in a wet state, e.g., having between about 10 percent and about 75 percent by weight water. The fiber source can even be mechanically treated while partially or fully submerged under a liquid, such as water, ethanol or isopropanol.

The fiber cellulosic or lignocellulosic material can also be mechanically treated under a gas (such as a stream or atmosphere of gas other than air), e.g., oxygen or nitrogen, or steam.

If desired, lignin can be removed from any of the fibrous materials that include lignin. Also, to aid in the breakdown of the materials that include cellulose, the material can be treated prior to or during mechanical treatment or irradiation with heat, a chemical (e.g., mineral acid, base or a strong oxidizer such as sodium hypochlorite) and/or an enzyme. For example, grinding can be performed in the presence of an acid.

Mechanical treatment systems can be configured to produce streams with specific morphology characteristics such as, for example, surface area, porosity, bulk density, and, in the case of fibrous feedstocks, fiber characteristics such as length-to-width ratio.

In some embodiments, a BET surface area of the mechanically treated material is greater than 0.1 m²/g, e.g., greater than 0.25 m²/g, greater than 0.5 m²/g, greater than 1.0 m²/g, greater than 1.5 m²/g, greater than 1.75 m²/g, greater than 5.0 m²/g, greater than 10 m²/g, greater than 25 m²/g, greater than 35 m²/g, greater than 50 m²/g, greater than 60 m²/g, greater than 75 m²/g, greater than 100 m²/g, greater than 150 m²/g, greater than 200 m²/g, or even greater than 250 m²/g.

A porosity of the mechanically treated material can be, e.g., greater than 20 percent, greater than 25 percent, greater than 35 percent, greater than 50 percent, greater than 60 percent, greater than 70 percent, greater than 80 percent, greater than 85 percent, greater than 90 percent, greater than 92 percent, greater than 94 percent, greater than 95 percent, greater than 97.5 percent, greater than 99 percent, or even greater than 99.5 percent.

In some embodiments, after mechanical treatment the material has a bulk density of less than 0.25 g/cm³, e.g., 0.20 g/cm³, 0.15 g/cm³, 0.10 g/cm³, 0.05 g/cm³ or less, e.g., 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters.

If the feedstock is a fibrous material the fibers of the fibrous materials mechanically treated material can have a relatively large average length-to-diameter ratio (e.g., greater than 20-to-1), even if they have been sheared more than once. In addition, the fibers of the fibrous materials described herein may have a relatively narrow length and/or length-to-diameter ratio distribution.

As used herein, average fiber widths (e.g., diameters) are those determined optically by randomly selecting approximately 5,000 fibers. Average fiber lengths are corrected length-weighted lengths. BET (Brunauer, Emmet and Teller) surface areas are multi-point surface areas, and porosities are those determined by mercury porosimetry.

If the second feedstock is a fibrous material 14 the average length-to-diameter ratio of fibers of the mechanically treated material can be, e.g. greater than 8/1, e.g., greater than 10/1, greater than 15/1, greater than 20/1, greater than 25/1, or greater than 50/1. An average fiber length of the mechanically treated material 14 can be, e.g., between about 0.5 mm and 2.5 mm, e.g., between about 0.75 mm and 1.0 mm, and an average width (e.g., diameter) of the second fibrous material 14 can be, e.g., between about 5 μm and 50 μm, e.g., between about 10 μm and 30 μm.

In some embodiments, if the feedstock is a fibrous material, the standard deviation of the fiber length of the mechanically treated material can be less than 60 percent of an average fiber length of the mechanically treated material, e.g., less than 50 percent of the average length, less than 40 percent of the average length, less than 25 percent of the average length, less than 10 percent of the average length, less than 5 percent of the average length, or even less than 1 percent of the average length.

Wet milling of the biomass feedstock can also be used as described in U.S. application Ser. No. 13/293,977 filed Nov. 10, 2011, the entire disclosure of which is herein incorporated by reference. For example a wet milling head using a rotor/stator can be used prior to the saccharification processes described herein. Alternatively wet milling can be done during the saccharification process. A system and method including jet milling, wet milling and the processes for saccharification described herein can also be used.

### Treatment to Solubilize, Reduce Recalcitrance or Functionalize

Materials that have or have not been physically prepared can be treated for use in any production process described herein. One or more of the production processes described below may be included in the recalcitrance reducing operating unit discussed above. Alternatively, or in addition, other processes for reducing recalcitrance may be included.

Treatment processes utilized by the recalcitrance reducing operating unit can include one or more of irradiation, sonication, oxidation, pyrolysis or steam explosion. Treatment methods can be used in combinations of two, three, four, or even all of these technologies (in any order).

### Radiation Treatment

One or more radiation processing sequences can be used to process materials from the feedstock, and to provide a wide variety of different sources to extract useful substances from the feedstock, and to provide partially degraded structurally modified material which functions as input to further processing steps and/or sequences. Irradiation can, for example, reduce the molecular weight and/or crystallinity of feedstock. Radiation can also sterilize the materials, or any media needed to bioprocess the material.

In some embodiments, energy deposited in a material that releases an electron from its atomic orbital is used to irradiate the materials. The radiation may be provided by (1) heavy charged particles, such as alpha particles or protons, (2) electrons, produced, for example, in beta decay or electron beam accelerators, or (3) electromagnetic radiation, for example, gamma rays, x rays, or ultraviolet rays. In one approach, radiation produced by radioactive substances can be used to irradiate the feedstock. In some embodiments, any combination in any order or concurrently of (1) through (3) may be utilized. In another approach, electromagnetic radiation (e.g., produced using electron beam emitters) can be used to irradiate the feedstock. The doses applied depend on the desired effect and the particular feedstock.

In some instances when chain scission is desirable and/or polymer chain functionalization is desirable, particles heavier than electrons, such as protons, helium nuclei, argon ions, silicon ions, neon ions, carbon ions, phosphorus ions, oxygen ions or nitrogen ions can be utilized. When ring-opening chain scission is desired, positively charged particles can be utilized for their Lewis acid properties for enhanced ring-opening chain scission. For example, when maximum oxidation is desired, oxygen ions can be utilized, and when maximum nitration is desired, nitrogen ions can be utilized. The use of heavy particles and positively charged particles is described in U.S. Pat. No. 7,931,784, the entire disclosure of which is herein incorporated by reference.

In one method, a first material that is or includes cellulose having a first number average molecular weight (M_{N1}) is irradiated, e.g., by treatment with ionizing radiation (e.g., in the form of gamma radiation, X-ray radiation, 100 nm to 280 nm ultraviolet (UV) light, a beam of electrons or other charged particles) to provide a second material that includes cellulose having a second number average molecular weight (M_{N2}) lower than the first number average molecular weight. The second material (or the first and second material) can be combined with a microorganism (with or without enzyme treatment) that can utilize the second and/or first material or its constituent sugars or lignin to produce an intermediate or a product, such as those described herein.

Since the second material includes cellulose having a reduced molecular weight relative to the first material, and in some instances, a reduced crystallinity as well, the second material is generally more dispersible, swellable and/or soluble, e.g., in a solution containing a microorganism and/or an enzyme. These properties make the second material easier to process and more susceptible to chemical, enzymatic and/or biological attack relative to the first material, which can greatly improve the production rate and/or production level of a desired product, e.g., ethanol. Radiation can also sterilize the materials or any media needed to bioprocess the material.

In some embodiments, the second material can have a level of oxidation (O₂) that is higher than the level of oxidation (O₁) of the first material. A higher level of oxidation of the material can aid in its dispersability, swellability and/or solubility, further enhancing the material's susceptibility to chemical, enzymatic or biological attack. In some embodiments, to increase the level of the oxidation of the second material relative to the first material, the irradiation is performed under an oxidizing environment, e.g., under a blanket of air or oxygen, producing a second material that is more oxidized than the first material. For example, the second material can have more hydroxyl groups, aldehyde groups, ketone groups, ester groups or carboxylic acid groups, which can increase its hydrophilicity.

### Ionizing Radiation

Each form of radiation ionizes the carbon-containing material via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired, positively charged particles may be desirable, in part due to their acidic nature. When particles are utilized, the particles can have the mass of a resting electron, or greater, e.g., 500, 1000, 1500, 2000, 10,000 or even 100,000 times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 amu. Accelerators used to accelerate the particles can be electrostatic DC, electrodynamic DC, RF linear, magnetic induction linear or continuous wave. For example, cyclotron type accelerators are available from IBA, Belgium, such as the Rhodotron® system, while DC type accelerators are available from RDI, now IBA Industrial, such as the Dynamitron®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy” Columbus-Ohio, ICRU-IAEA Meeting, 18-20 March 2006, Iwata, Y. et al., “Alternating-Phase-Focused IH-DTL for Heavy-Ion Medical Accelerators” Proceedings of EPAC 2006, Edinburgh, Scotland and Leaner, C. M. et al., “Status of the Superconducting ECR Ion Source Venus” Proceedings of EPAC 2000, Vienna, Austria.

In some embodiments, a beam of electrons is used as the radiation source. A beam of electrons has the advantages of high dose rates (e.g., 1, 5, or even 10 Mrad per second), high throughput, less containment, and less confinement equipment. Electrons can also be more efficient at causing chain scission. In addition, electrons having energies of 4-10 MeV can have a penetration depth of 5 to 30 mm or more, such as 40 mm. In some cases, multiple electron beam devices (e.g., multiple heads, often referred to as “horns”) are used to deliver multiple doses of electron beam radiation to the material. This high total beam power is usually achieved by utilizing multiple accelerating heads. For example, the electron beam device may include two, four, or more accelerating heads. As one example, the electron beam device may include four accelerating heads, each of which has a beam power of 300 kW, for a total beam power of 1200 kW. The use of multiple heads, each of which has a relatively low beam power, prevents excessive temperature rise in the material, thereby preventing burning of the material, and also increases the uniformity of the dose through the thickness of the layer of material. Irradiating with multiple heads is disclosed in U.S. application Ser. No. 13/276,192 filed Oct. 18, 2011, the complete disclosure of which is incorporated herein by reference.

Electron beams can be generated, e.g., by electrostatic generators, cascade generators, transformer generators, low energy accelerators with a scanning system, low energy accelerators with a linear cathode, linear accelerators, and pulsed accelerators. Electrons as an ionizing radiation source can be useful, e.g., for relatively thin piles of materials, e.g., less than 0.5 inch, e.g., less than 0.4 inch, 0.3 inch, 0.2 inch, or less than 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV.

Electron beam irradiation devices may be procured commercially from Ion Beam Applications, Louvain-la-Neuve, Belgium or the Titan Corporation, San Diego, Calif. Typical electron energies can be 1 MeV, 2 MeV, 4.5 MeV, 7.5 MeV, or 10 MeV. Typical electron beam irradiation device power can be 1 kW, 5 kW, 10 kW, 20 kW, 50 kW, 100 kW, 250 kW, or 500 kW. The level of depolymerization of the feedstock depends on the electron energy used and the dose applied, while exposure time depends on the power and dose. Typical doses may take values of 1 kGy, 5 kGy, 10 kGy, 20 kGy, 50 kGy, 100 kGy, or 200 kGy. In a some embodiments energies between 0.25-10 MeV (e.g., 0.5-0.8 MeV, 0.5-5 MeV, 0.8-4 MeV, 0.8-3 MeV, 0.8-2 MeV or 0.8-1.5 MeV) can be used.

### Electromagnetic Radiation

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ Hz, greater than 10¹⁷ Hz, 10¹⁸, 10¹⁹, 10²⁰, or even greater than 10²¹ Hz. In some embodiments, the electromagnetic radiation has a frequency of between 10¹⁸ and 10²² Hz, e.g., between 10¹⁹ to 10²¹ Hz

### Doses

In some embodiments, the irradiating (with any radiation source or a combination of sources) is performed until the material receives a dose of at least 0.25 Mrad, e.g., at least 1.0, 2.5, 5.0, 8.0, 10, 15, 20, 25, 30, 35, 40, 50, or even at least 100 Mrad. In some embodiments, the irradiating is performed until the material receives a dose of between 1.0 Mrad and 6.0 Mrad, e.g., between 1.5 Mrad and 4.0 Mrad, 2 Mrad and 10 Mrad, 5 Mrad and 20 Mrad, 10 Mrad and 30 Mrad, 10 Mrad and 40 Mrad, or 20 Mrad and 50 Mrad.

In some embodiments, the irradiating is performed at a dose rate of between 5.0 and 1500.0 kilorads/hour, e.g., between 10.0 and 750.0 kilorads/hour or between 50.0 and 350.0 kilorads/hours.

In some embodiments, two or more radiation sources are used, such as two or more ionizing radiations. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light.

### Sonication, Pyrolysis and Oxidation

In addition to radiation treatment, the feedstock may be treated with any one or more of sonication, pyrolysis and oxidation. These treatment processes are described in U.S. Pat. No. 7,932,065 filed Apr. 23, 2009, the entire disclosure of which is herein incorporated by reference.

### Other Processes to Solubilize, Reduce Recalcitrance or to Functionalize

Any of the processes of this paragraph can be used alone without any of the processes described herein, or in combination with any of the processes described herein (in any order): steam explosion, chemical treatment (e.g., acid treatment (including concentrated and dilute acid treatment with mineral acids, such as sulfuric acid, hydrochloric acid and organic acids, such as trifluoroacetic acid), and/or base treatment (e.g., treatment with lime or sodium hydroxide), UV treatment, screw extrusion treatment, solvent treatment (e.g., treatment with ionic liquids) and freeze milling. Some of these processes, for example, are described in U.S. Pat. No. 8,063,201 filed Nov. 19, 2010 and; U.S. application Ser. No. 13/099,151 filed May 2, 2011; and U.S. Pat. No. 7,900,857 filed Jul. 14, 2009, the entire disclosures of which are herein incorporated by reference.

### Products and Post Saccharification Processing

### Sugars

Processing during or after saccharification can include isolation and/or concentration of sugars by chromatography e.g., simulated moving bed chromatography, precipitation, centrifugation, crystallization, solvent evaporation and combinations thereof. In addition, or optionally, processing can include isomerization of one or more of the sugars in the sugar solution or suspension.

Some possible processing steps are disclosed in PCT/US12/71093, PCT/US12/71083 and PCT/US 12/71097 filed on Dec. 20, 2012 the entire disclosures of which are herein incorporated by reference.

### Hydrogenation

Downstream processing can include hydrogenation. For example glucose and xylose can be hydrogenated to sorbitol and xylitol respectively. Hydrogenation can accomplished by use of a catalyst e.g., Pt/γ-Al₂O₃, Ru/C, Raney Nickel in combination with H₂ under high pressure e.g., 10 to 12000 psi.

### Fuel Cells

Where the methods described herein produce a sugar solution or suspension, this solution or suspension can subsequently be used in a fuel cell. For example, fuel cells utilizing sugars derived from cellulosic or lignocellulosic materials are disclosed in PCT/US 12/70624 filed Dec, 19, 2012, the entire disclosure of which is herein incorporated by reference. Fermentation

In downstream processing, the sugars produced by saccharification can be fermented to produce other products, e.g., alcohols, sugar alcohols, such as erythritol, organic acids, e.g., lactic, glutamic or citric acids or amino acids.

Yeast and Zymomonas bacteria, for example, can be used for fermentation. Other microorganisms are discussed in the Materials section, below.

The optimum pH for yeast is from about pH 4 to 5, while the optimum pH for Zymomonas is from about pH 5 to 6. Typical fermentation times are about 24 to 96 hours with temperatures in the range of 26° C. to 40° C., however thermophilic microorganisms prefer higher temperatures.

In some embodiments e.g., when anaerobic organisms, for example Clostridia, are used, at least a portion of the fermentation is conducted in the absence of oxygen e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic condition can be achieved or maintained by carbon dioxide production during the fermentation and no additional inert gas is needed.

Jet mixing may be used during fermentation, and in some cases saccharification and fermentation are performed in the same tanks, simultaneously or sequentially.

Nutrients may be added during saccharification and/or fermentation, for example the food-based nutrient packages described in U.S. application Ser. No. 13/184,138 filed Jul. 15, 2011, the entire disclosure of which is incorporated herein by reference.

Mobile fermentors can be utilized, as described in U.S. Ser. No. 12/374,549 and International Application No. WO 2008/011598. Similarly, the saccharification equipment can be mobile. Further, saccharification and/or fermentation may be performed in part or entirely during transit.

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Intermediates and Products

Specific examples of products that may be produced utilizing the processes disclosed herein include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols, e.g., containing greater than 10%, 20%, 30% or even greater than 40% water, xylitol, biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives, e.g., fuel additives. Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha, beta unsaturated acids, such as acrylic acid and olefins, such as ethylene. Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), methyl or ethyl esters of any of these alcohols. Other products include methyl acrylate, methylmethacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, y-hydroxybutyric acid, and mixture thereof, a salt of any of these acids, or a mixture of any of the acids and their respective salts. a salt of any of the acids and a mixture of any of the acids and respective salts

Other intermediates and products, including food and pharmaceutical products, are described in U.S. Ser. No. 12/417,900 filed Apr. 3, 2009, the entire disclosure of which is herein incorporated by reference. Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be irradiated prior to selling the products, e.g., after purification or isolation or even after packaging, for example to sanitize or sterilize the product(s) and/or to neutralize one or more potentially undesirable contaminants that could be present in the product(s). Such irradiation may, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

The spent biomass from lignocellulosic processing by the methods described are expected to have a high lignin content and may be a valuable product. For example, the lignin can be used as captured as a plastic, or it can be synthetically upgraded to other plastics. In some instances, it can be utilized as an energy source, e.g., burned to provide heat. In some instances, it can also be converted to lignosulfonates, which can be utilized as binders, dispersants, emulsifiers or as sequestrants.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

As a dispersant, the lignin or lignosulfonates can be used, e.g., concrete mixes, clay and ceramics, dyes and pigments, leather tanning and in gypsum board.

As an emulsifier, the lignin or lignosulfonates can be used, e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

As a heating source, lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

### Feedstock Materials

### Biomass Feedstock

The feedstock is preferably a lignocellulosic material, although the processes described herein may also be used with cellulosic materials, e.g., paper, paper products, paper pulp, cotton, and mixtures of any of these, and other types of biomass. The processes described herein are particularly useful with lignocellulosic materials, because these processes are particularly effective in reducing the recalcitrance of lignocellulosic materials and allowing such materials to be processed into products and intermediates in an economically viable manner.

In some cases, the lignocellulosic material can include, for example, wood, grasses, e.g., switchgrass, grain residues, e.g., rice hulls, bagasse, jute, hemp, flax, bamboo, sisal, abaca, straw, corn cobs, corn stover, coconut hair, algae, seaweed, wheat straw and mixtures of any of these.

In some cases, the lignocellulosic material includes corncobs. Ground or hammermilled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or feedstocks containing significant amounts of corncobs.

Corncobs, before and after comminution, are also easier to convey and disperse, and have a lesser tendency to form explosive mixtures in air than other feedstocks such as hay and grasses.

Other sources of cellulosic or lignocellulosic materials are from genetically modified plants is disclosed in U.S. application Ser. No. 13/396,369 filed Feb. 14, 2012 the complete disclosure of which is incorporated herein by reference.

Other biomass feedstocks include starchy or sugary materials and microbial materials.

Starchy or sugary materials include starch itself, e.g., corn starch, wheat starch, potato starch or rice starch, a derivative of starch, or a material that includes starch or sugar, such as an edible food product or a crop. For example, the starchy or sugary material can be arracacha, buckwheat, banana, barley, cassava, kudzu, oca, sago, sorghum, regular household potatoes, sweet potato, taro, yams, corn kernels, or one or more beans, such as favas, lentils or peas. Blends of any two or more starchy or sugary materials are also starchy/sugary materials.

Microbial sources include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture systems.

Blends of any biomass materials described herein can be utilized for making any of the intermediates or products described herein. For example, blends of cellulosic materials and starchy materials can be utilized for making any product described herein.

### Saccharifying Agents

Suitable cellulolytic enzymes include cellulases from the genera Bacillus, Pseudomonas, Humicola, Fusarium, Thielavia, Acremonium, Chrysosporium and Trichoderma, and include species of Humicola, Coprinus, Thielavia, Fusarium, Myceliophthora, Acremonium, Cephalosporium, Scytalidium, Penicillium or Aspergillus (see, e.g., EP 458162), especially those produced by a strain selected from the species Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additionally, Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, e.g., EP 458162) may be used.

### Fermentation Agents

The microorganism(s) used in fermentation can be natural microorganisms and/or engineered microorganisms. For example, the microorganism can be a bacterium, e.g., a cellulolytic bacterium, a fungus, e.g., a yeast, a plant or a protist, e.g., an algae, a protozoa or a fungus-like protist, e.g., a slime mold. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Sacchromyces spp. e.g., Sacchromyces cerevisiae (baker's yeast), Saccharomyces distaticus, Saccharomyces uvarum; the genus Kluyveromyces, e.g., species Kluyveromyces marxianus, Kluyveromyces fragilis; the genus Candida, e.g., Candida pseudotropicalis, and Candida brassicae, Pichia stipitis (a relative of Candida shehatae, the genus Clavispora, e.g., species Clavispora lusitaniae and Clavispora opuntiae, the genus Pachysolen, e.g., species Pachysolen tannophilus, the genus Bretannomyces, e.g., species Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212). Other suitable microorganisms include, for example, Zymomonas mobilis, Clostridium thermocellum (Philippidis, 1996, supra), Clostridium saccharobutylacetonicum, Clostridium saccharobutylicum, Clostridium Puniceum, Clostridium beijernckii, Clostridium acetobutylicum, Moniliella pollinis, Yarrowia lipolytica, Aureobasidium sp., Trichosporonoides sp., Trigonopsis variabilis, Trichosporon sp., Moniliellaacetoabutans, Typhula variabilis, Candida magnoliae, Ustilaginomycetes, Pseudozyma tsukubaensis, yeast species of genera Zygosaccharomyces, Debaryomyces, Hansenula and Pichia, and fungi of the dematioid genus Torula.

Commercially available yeasts include, for example, Red Star®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Glucose Isomerase

Glucose isomerase (also known as xylose isomerase and D-xylose ketol isomerase) belongs to a family of isomerases that interconvert aldoses and ketoses. Some examples are the isomerases (EC 5.3.19), (EC 5.3.16) and EC 5.3.1.5.

The glucose isomerize used can be isolated from many bacteria including but not limited to: Actinomyces olivocinereus, Actinomyces phaeochromogenes, Actinoplanes missouriensis, Aerobacter aerogenes, Aerobacter cloacae, Aerobacter levanicum, Arthrobacter spp., Bacillus stearothermophilus, Bacillus megabacterium, Bacillus coagulans, Bifidobacterium spp., Brevibacterium incertum, Brevibacterium pentosoaminoacidicum, Chainia spp., Corynebacterium spp., Cortobacterium helvolum, Escherichia freundii, Escherichia intermedia, Escherichia coli, Flavobacterium arborescens, Flavobacterium devorans, Lactobacillus brevis, Lactobacillus buchneri, Lactobacillus fermenti, Lactobacillus mannitopoeus, Lactobacillus gayonii, Lactobacillus plantarum, Lactobacillus lycopersici, Lactobacillus pentosus, Leuconostoc mesenteroides, Microbispora rosea, Microellobosporia flavea, Micromonospora coerula, Mycobacterium spp., Nocardia asteroides, Nocardia corallia, Nocardia dassonvillei, Paracolobacterium aerogenoides, Pseudonocardia spp., Pseudomonas hydrophila, Sarcina spp., Staphylococcus bibila, Staphylococcus flavovirens, Staphylococcus echinatus, Streptococcus achromogenes, Streptococcus phaeochromogenes, Streptococcus fracliae, Streptococcus roseochromogenes, Streptococcus olivaceus, Streptococcus californicos, Streptococcus venuceus, Streptococcus virginial, Streptomyces olivochromogenes, Streptococcus venezaelie, Streptococcus wedmorensis, Streptococcus griseolus, Streptococcus glaucescens, Streptococcus bikiniensis, Streptococcus rubiginosus, Streptococcus achinatus, Streptococcus cinnamonensis, Streptococcus fradiae, Streptococcus albus, Streptococcus griseus, Streptococcus hivens, Streptococcus matensis, Streptococcus murinus, Streptococcus nivens, Streptococcus platensis, Streptosporangium album, Streptosporangium oulgare, Thermopolyspora spp., Thermus spp., Xanthomonas spp. and Zymononas mobilis.

Glucose isomerase can be used free in solution or immobilized on a support. Whole cells or cell free enzymes can be immobilized. The support structure can be any insoluble material. Support structures can be cationic, anionic or neutral materials, for example diethylaminoethyl cellulose, metal oxides, metal chlorides, metal carbonates and polystyrenes. Immobilization can be accomplished by any suitable means. For example immobilization can be accomplished by contacting the support and the whole cell or enzyme in a solvent such as water and then removing the solvent. The solvent can be removed by any suitable means, for example filtration or evaporation or spray drying. As another example, spray drying the whole cells or enzyme with a support can be effective. Glucose isomerase can also be present in a living cell that produces the enzyme during the process.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method comprising:
separating a solid saccharified biomass from a liquid medium, and
saccharifying the solid saccharified biomass.

2. The method of claim 1 wherein the liquid medium comprises enzymes and sugars.

3. The method of claim 1 wherein the solid saccharified biomass is wetted by the liquid medium.

4. The method of claim 1 wherein the solid saccharified biomass and liquid medium are produced by saccharifiying a solid biomass in a liquid.

5. The method of claim 4 wherein the biomass has been treated by a method selected from the group consisting of irradiation, sonication, oxidation, pyrolysis, steam explosion and combinations thereof.

6. The method claim 4 wherein the biomass has been treated by irradiation.

7. The method of claim 6 wherein the biomass receives a total dosage of between about 10 and 200 Mrad.

8. The method of claim 1 wherein the solid saccharified biomass and liquid medium are separated by a separator selected from the group consisting of a centrifuge, a filtering device, a settling tank, a porous material, a mesh, a strainer, a vibratory screener, a perforated plate or cylinder, a sieving device and combinations of these.

9. The method of claim 4 wherein saccharifiying the biomass is completed.

10. The method of claim 4 wherein saccharifiying the biomass is at least 20% completed.

11. The method of claim 4 wherein the biomass is a cellulosic or lignocellulosic biomass.

12. The method of claim 11 wherein the biomass is selected form the group consisting of paper, paper products, paper waste, wood, particle board, sawdust, agricultural waste, sewage, silage, grasses, straw, wheat straw, rice hulls, bagasse, cotton, jute, hemp, flax, bamboo, sisal, abaca, straw, corn cobs, corn stover, alfalfa, hay, coconut hair, seaweed, algae, and mixtures thereof.

13. The method of claim 4 wherein saccharifying is done using at least one jet mixer.

14. A method comprising:
saccharifying a solid biomass in a liquid;
separating solid saccharified biomass from the liquid;
removing the liquids from the separated saccharified biomass, and
adding liquid and a saccharifiying agent to separated saccharified biomass.

15. The method of claim 14 wherein saccharifying the biomass material is done while mixing the solid biomass material in a liquid using a mixer.

16. The method of claim 15 wherein mixing is done using at least one jet mixer.

17. The method of claim 15 wherein separating is done by allowing the solid saccharified biomass to settle and decanting the liquids from the solid.

18. The method of claim 14 wherein separating is done by using a continuous centrifuge.

19. The method of claim 14 wherein separating is done by using a settling tank.
